(19) United States
(12) Patent Application Publication
(10) Pub. No.: US 2014/0154784 A1
(43) Pub. Date: Jun. 5, 2014

(54) Method and Process for the Degradation of Cyclic Ethers in Ethoxylate-Containing Actives

(71) Applicant: Gregory van Buskirk, Danville, CA (US)

(72) Inventor: Gregory van Buskirk, Danville, CA (US)

(73) Assignee: Gregory van Buskirk

(21) Appl. No.: 13/692,982

(22) Filed: Dec. 3, 2012

(51) Int. Cl. C07C 303/44

(52) U.S. Cl. CPC C07C 303/44
USPC 435/280; 568/622

(57) ABSTRACT

The invention provides a method and process for the degradation of cyclic ethers that are often found in liquid streams comprising cleaning actives, through the addition of cyclic ether-degrading enzymes or enzyme-producing microbes. The cleaning actives that may be successfully treated by the methods described are selected from among anionic surfactants, nonionic surfactants, solvents, derivatives thereof, as well as mixtures of any of the foregoing.

### BACKGROUND OF THE INVENTION

Surface-active agents, also known as surfactants, are primary ingredients of cleaning product formulations. Surfactants are usually organic amphiphilic compounds, meaning that they are composed of a hydrophobic or water-fearing fatty portion, and a hydrophilic or water-loving portion. Examples of groups that provide hydrophilicity to surfactants include carboxylates (RCO₂⁻), sulfates (RSO₄⁻), sulfonates (RSO₃⁻), phosphates (RPO₄²⁻), amines (RNH₃⁺), and polyoxyethylene glycols. The latter are also known as polyethylene oxides, or Eos and may be represented as: R—(OCH₂CH₂)ₙ—OH. Worldwide consumption of surfactants has been estimated at 15 million tons (30 billion pounds) per year (Kosswig, K. “Surfactants” in Ullmann's Encyclopedia of Industrial Chemistry, Wiley-VCH, 2005, Weinheim).

Ethoxylated surfactants and their derivatives, examples of which include alkyl ether sulfates, alkyl ether carboxylates and alkyl ether phosphates, are very useful cleaning agents. They share in common the presence of at least one ethylene oxide unit, and are therefore considered ether derivatives. They are used worldwide for their cleansing efficacy, biodegradability and relative mildness. Worldwide usage of alcohol ethoxylates ranges between an estimated 1.7 million metric tons (3.7 billion pounds) in 2003 (see Brackmann, B. and C.-D. Hager, “The Statistical World of Raw materials, Fatty Alcohols, and Surfactants,” CD Proceedings 6^{th} World Surfactant Congress CESIO, Berlin Germany, June 2004, paper #112) to an estimated 612,000 metric tons (1.35 billion pounds) in 2009 (Janshekar, H., S. Rizvi, and Y. Inoguchi, “CEH Marketing Research Report: Surfactants Household Detergents and Their Raw Materials,” Chemical Economics Handbook, SRI Consulting, June 2010, p. 11).

During the production of ethoxylated surfactants and their derivatives, a common side reaction results in the generation of dioxanes, which are produced as byproducts of the ethoxylation process. Dioxanes are cyclic diethers, and while dioxane has three isomers, the 1,2-dioxacyclohexane and 1,3-dioxacyclohexane forms are rare. Consequently, the term dioxane is typically understood to primarily refer to the 1,4-dioxyacyclohexane isomer, which is also known as diethylene oxide, diethylene dioxide, diethylene ether, dioxyethylene ether, glycol ethylene ether, and p-dioxane.

Dioxane is used primarily as a stabilizer for 1,1,1-trichloroethane. This heterocyclic organic compound is also often used as a solvent in the laboratory, as well as in inks and adhesives. 1,4-dioxane is formed when diethylene glycol, which is generated during the hydrolysis of ethylene oxide, is dehydrated in the presence of an acid catalyst. As ether sulfates, ether carboxylates and ether phosphates are all produced under acidic conditions, 1,4-dioxane is a ubiquitous contaminant. It is stable and very water soluble, thus making it difficult to remove from aqueous solutions. Moreover, 1,4-dioxane is resistant to natural biodegradation processes.

The amounts of 1,4 dioxane that can be found in surfactant compositions as well as in commercial cleaning products can vary widely. The levels can vary from parts per billion to percents. While usually present in amounts low enough to avoid safety concerns, 1,4-dioxane is nevertheless a chemical that requires attention, as it is classified as a Group 2B carcinogen by the International Agency for Research on Cancer (IARC) because it is a known carcinogen in animals. Environmental and safety groups have repeatedly found the chemical to be present in consumer products, and subsequently warn consumers from using said products. Given the tonnage of surfactants in commerce that contain 1,4-dioxane, elimination of the contaminant is a high priority concern.

Currently, manufacturers of ethoxylated surfactants attempt to avoid formation of 1,4-dioxane during surfactant production through rigorous controls of starting materials and processing conditions. However, even the best manufacturing processing cannot avoid some formation of 1,4-dioxane. Consequently, further effort is often expended in removing 1,4-dioxane from surfactants after dioxane has formed. The removal process involves such techniques as vacuum stripping and flushing surfactant mixtures with nitrogen gas to vaporize 1,4-dioxane from the mixture. The problem with these dioxane removal techniques is that they are additional, time-consuming manufacturing steps, which are neither efficient nor cost-effective. Moreover, these methods can result in excessive foaming by the surfactant during volatilization of the 1,4-dioxane. Heating of the mixture in an attempt to reduce foaming can result in degradation of the surfactant mixture. In yet another removal method, 1,4-dioxane can be reduced by partitioning it into another organic solvent that preferentially extracts the 1,4-dioxane; this necessitates subsequent removal of the solvent, which can also be difficult and expensive.

A number of publications discuss the use of microbes and/or enzymes to remediate 1,4-dioxane once it has entered the environment. Several examples of using microbes and/or enzymes in this manner are discussed here. Parales, et al., isolated an acetomycete (CB1190) from a dioxane-contaminated sludge sample. This bacterium was capable of sustained aerobic growth on 1,4-dioxane, as well as on other cyclic and linear ethers. See Parales, R. E., J. E. Adamus, and H. D. May, “Degradation of 1,4-Dixoane by an Actinomycete in Pure Culture”, Applied and Environmental Microbiology, 60(12), 1994 (pp 4527-4530).

In another article, Kinne, et al., found that the extracellular peroxygenase of the fungus Agrocybe aegerita catalyzed the H₂O₂-dependent cleavage of environmentally significant ethers, including methyl t-butyl ether, tetrahydrofuran, and 1,4-dioxane. The authors noted that the ether cleavage was not specific. Indeed, they noted that ether linkages are widespread in soils and litter, not only in abundant natural substances such as lignin, flavonoids, and lignans, but also in anthropogenic compounds that include many solvents, biocides, and surfactants. Further, it was speculated that an oxidative mechanism is required for the biodegradation of ethers, which are relatively recalcitrant because they do not hydrolyze at physiological pH values. See Kinne , M., M. Poraj-Kobielska, S. A. Ralph, R. Ullrich, M. Hofrichter, and K. E. Hammel, “Oxidative Cleavage of Diverse Ethers by a Fungal Peroxygenase”, Journal of Biological Chemistry, 284(43), 1999 (pp 29343-29349).

In 2001, Kelley, et al., investigated the potential to enhance dioxane biodegradation in both planted and unplanted soil, by adding the dioxane-degrading actinomycete. Poplar root extract stimulated dioxane degradation in bioaugmented soil, as did tetrahydrofuran (THF) and 1-butanol which acted as co-substrates that enhanced dioxane degradation by CB1190. Glucose and soil extract did not affect dioxane degradation. See Kelley, S. L., E. W. Aitchison, M. Deshpande, J. L. Schnoor and P. J. J. Alvarez, “Biodegradation of 1,4-Dioxane in Planted and Unplanted Soil: Effect of Bioaugmentation With Amycolata sp. CB1190”, Water Res., 35(16), 2001 (pp 3791-3800).

In another journal article, Nakamiya, et al., isolated the fungus Cordyceps sinesis, which was found to be capable of degrading 1,4-dioxane. While the actual dioxane-degrading enzyme(s) was not isolated, it was postulated that etherases and/or oxidases were the substances likely responsible for the degradation of 1,4-dioxane. See Nakamiya, K., S, Hashimoto, H. Ito, J. S. Edmonds, and M. Morita, 2005, “Degradation of 1,4-Dioxane and Cyclic Ethers by an Isolated Fungus”, Applied and Environmental Microbiology, 71(3), 2005 (pp 1254-1258).

Mahendra, et al., isolated 13 bacterial samples capable of transforming dioxane, including Pseudonocardia dioxanivorans CB1190, Pseudonocardia benzenivorans B5, Pseudonocardia K1, Methylosinus trichosporium OB3b, Mycobacterium vaccae JOB5, Rhodococcus RR1, Burkholderia cepacia G4, Ralstonia pickettii PKO1, Pseudomonas mendocina KR1, Escherichia coli TG1(T2MO), Escherichia coli TG1(TpMO), and Escherichia coli TG1(T4MO). The first two bacterial isolates (Pseudonocardia dioxanivorans CB1190 and Pseudonocardia benzenivorans B5) were able to grow where 1,4-dioxane was the sole source of carbon. See Mahendra, S. and L. Alvarez-Cohen, 2006, “Kinetics of 1,4-Dioxane Biodegradation by Monooxygenase-Expressing Bacteria”, Environmental Science and Technology, 40(17), 2006 (pp 5435-5442).

More recently, Bioremediation Consulting Incorporated (BCI) reported the culturing of propane-utilizing aerobic bacteria, or propanotrophs, from groundwater at several sites. All cultures apparently have the ability to co-metabolically degrade propane and 1,4-dioxane using the enzyme system designed to oxidize propane. Generally, dioxane and propane ‘compete’ for oxygen such that dioxane is degraded when aqueous propane has been nearly depleted. BCI reported that concentrations of dioxane up to about 10 ppm are readily degraded, but higher concentrations are inhibitory. For in situ bioremediation of dioxane, groundwater would be amended with propane and probably also oxygen. See
http://www.bcilabs.com/dioxane_degrading.html, 3 Dec. 2012.

While microorganisms and/or enzymes have been described that effect degradation of 1,4-dioxane in model environmental systems, none of the foregoing articles address the removal of dioxanes from products prior to their use by consumers or their subsequent introduction into the environment. Furthermore, none of the foregoing references address the removal or reduction of 1,4-dioxane in the presence of other ether derivatives such as ethoxylated surfactants.

### SUMMARY OF THE INVENTION

The focus of the prior art has been to decrease levels of contaminates such as 1,4-dioxane that is found in soil or groundwater by using microbes to degrade the contaminant once it is found in the environment. To date, however, no reference has been found that either relates to the removal of dioxanes from raw materials such as ethoxylated surfactants that are used in cleaning product formulations, or that describe the use of microbes in processes for making cleaning products or the like. In other words, it is not until after it enters the environment that bioremediation using microorganisms is used—either in situ or ex situ—to degrade dioxanes. The problem with this approach is that consumers can be exposed to 1,4-dioxane through the use of cleaning products. Post-usage, therefore dioxanes may already have entered and become dissipated into the environment before a treatment technique is applied.

The present invention therefore provides an improvement over prior art approaches to dioxane bioremediation and offers the benefit of providing a technique for the removal of dioxanes and cyclic ethers before they are encountered by consumers or before they can enter the environment. Accordingly, the present invention involves the use of microbes and enzymes for use in degrading 1,4-dioxane and cyclical ethers either as they are formed or prior to the delivery of surfactant-containing compositions to a consumer or other end user of the composition. As can be appreciated by those knowledgeable in the relevant field, reference to such surfactant-containing compositions herein is also understood to include ethoxylated surfactants, solvents, and their respective derivatives.

### DETAILED DESCRIPTION OF THE PREFERRED EMBODIMENTS

The present invention teaches methods for the removal of 1,4-dioxane from cleaning products, solvents and their derivatives before they are encountered by consumers or other end users, before they can enter the environment. More specifically, the present invention teaches the removal of dioxane from raw materials such as surfactants, solvents and their derivatives; from cleaning products that contain surfactants and solvents and their derivatives; and from other products that contain either 1,4-dioxane or cyclic ethers that may form dioxanes in the presence of an acid catalyst. For illustrative purposes, a general scheme for the formation of dioxanes from cyclic ethers such as ethylene oxide is shown in Scheme 1:

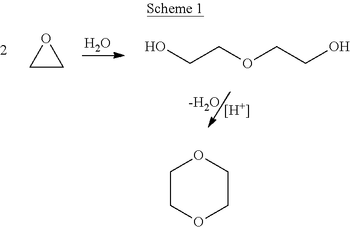

While the specific dioxane shown in Scheme 1 is 1,4-dioxane, it will be understood by knowledgeable practitioners in the relevant art that Scheme 1 is illustrative only, and that various isomeric forms of glycol ethers and thus additional forms of dioxanes may result.

The range of surfactants and solvents envisioned for the inventive treatment include which, by virtue of ethylene oxide groups, have at least some measurable initial level of 1,4-dioxane. This would include nonionic surfactants (such as alcohol ethoxylates, alcohol ethoxylate propoxylates, alkyl ethanolamide ethoxylates and amine ethoxylates) as well as anionic surfactants (such as alkyl ether sulfates, alkyl ether carboxylates, and alkyl ether phosphates). While measurable amounts of 1,4-dioxane have been found in alcohol ethoxylates, the levels are usually lower than those found for the other surfactants. Indeed, commercial samples of polyalkoxylated polyamine products were found to contain between 1-2% of 1,4-dioxane by weight. In addition, typical levels of 1,4-dioxane in alkyl ether sulfates are about 25-100 ppm, quite enough to measured in commercial cleaning products despite dilution levels.

### Nonionic Surfactants

Nonionic surfactants that are amenable to the methods and processes described herein are preferably selected from the group consisting of: C₆₋₁₈ alcohols that contain 1-15 moles of ethylene oxide per mole of alcohol; C₆₋₁₈ alcohols with 1-15 moles of ethylene oxide and 1-10 moles of propylene oxide per mole of alcohol; C₆₋₁₈ alkylphenols with 1-15 moles of ethylene oxide or propylene oxide or both per mole of alcohol; and mixtures of any of the foregoing. Several surfactants are available from Shell Chemical Company under the trademark Neodol. Included among suitable surfactants is Neodol 25-9, a C₁₂₋₁₅ alcohol with an average of nine moles of ethylene oxide per mole of alcohol. Another suitable surfactant is Surfonic L12-6, which is a C₁₀₋₁₂ alcohol, ethoxylated with about six moles of ethylene oxide per mole of alcohol, available from Huntsman Performance Products (Houston, Tex.). These and other nonionic surfactants suitable for use with the invention can be linear or branched primary or secondary alcohols, amides, or amines. Partially unsaturated surfactants that may be treated according to the present invention can vary from C₁₀₋₂₂ alkoxylated alcohols, with a minimum iodine value of at least 40, an example of which is described by Drozd, et al., U.S. Pat. No. 4,668,423. An example of an ethoxylated, propoxylated alcohol is Surfonic JL-80X, a C₉₋₁₁ alcohol having about 9 moles of ethylene oxide and 1.5 moles of propylene oxide per mole of alcohol, also available from Huntsman Performance Products.

Other suitable nonionic surfactants may include polyoxyethylene carboxylic acid esters, ethoxylated fatty acid alkanolamides, ethoxylated fatty amines; certain block copolymers of propylene oxide and ethylene oxide and block polymers of propylene oxide and ethylene oxide with a propoxylated ethylene diamine or some other suitable initiator.

### Anionic Surfactants

The anionic surfactants are preferably selected from the group consisting of alkyl ether sulfates, alkyl ether carboxylates, and alkyl ether phosphates. In general, the use of such ethoxylated anionic surfactants is preferred since, unlike other anionic surfactants, e.g., alkyl benzene sulfonate (LAS), they exhibit very good foaming, are milder to skin contact, and have less deleterious effects on cleaning enzymes. More importantly, unlike regular fatty acid soaps or LAS, phase instability because of co-precipitation with the calcium salts is less. They are important surfactants, both in their acidic and their neutralized form.

The alkyl ether sulfate, also known as an alcohol alkoxysulfate, is preferably a C₈₋₁₈, more preferably C₁₀₋₁₆, and most preferably C₁₂₋₁₄, fatty alcohol, which has been ethoxylated with an average of about 1-20, more preferably 2-15, and most preferably 3-10 moles of ethylene oxide per mole of alcohol, and subsequently sulfonated. These are also known as sulfonated fatty alcohol ethoxylates. It is preferred that if a mixture of fatty alcohols is used, that the higher molecular weight portions (i.e., C₁₄ and greater) are present in lesser amounts, although higher alkyl ether sulfates may be utilized by having higher amounts of ethylene oxide to aid in dispersing the compound in aqueous solution. An especially preferred alkyl ether carboxylate is Steol CS-270 C, a C₁₂ fatty alcohol ether sulfate with an average 2 moles of ethylene oxide per mole of alcohol, which is available from Stepan Company.

The alkyl ether carboxylate, also known as an alcohol alkoxycarboxylate, is preferably a C₈₋₁₈, more preferably C₁₀₋₁₆, and most preferably C₁₂₋₁₄, fatty alcohol, which has been ethoxylated with an average of about 1-20, more preferably 2-15, and most preferably 3-10 moles of ethylene oxide per mole of alcohol, and subsequently carboxylated. These are also known as carboxylated fatty alcohol ethoxylates. It is preferred that if a mixture of fatty alcohols is used, that the higher molecular weight portions (i.e., C₁₄ and greater) are present in lesser amounts, although higher alkyl ether carboxylates may be utilized by having higher amounts of ethylene oxide to aid in dispersing the compound in aqueous solution. An especially preferred alkyl ether carboxylate is Sandopan DTC, a C₁₃ fatty alcohol ether carboxylate with an average 7 moles of ethylene oxide per mole of alcohol, which is available from Clariant (North Carolina).

The alkyl ether phosphate, also known as an alcohol alkoxyphosphate, is preferably a C₈₋₁₈, more preferably C₁₀₋₁₆, and most preferably C₁₂₋₁₄, fatty alcohol, which has been ethoxylated with an average of about 1-20, more preferably 2-15, and most preferably 3-10 moles of ethylene oxide per mole of alcohol, and subsequently phosphated. They are also known as phosphated fatty alcohol ethoxylates. It is preferred that if a mixture of fatty alcohols is used, that the higher molecular weight portions (i.e., C₁₄ and greater) are present in lesser amounts, although higher alkyl ether carboxylates may be utilized by having higher amounts of ethylene oxide to aid in dispersing the compound in aqueous solution. An especially preferred alkyl ether phosphate is Slovafos 4, a C₁₂₋₁₄ fatty alcohol ether phosphate with an average of four moles of ethylene oxide per mole of alcohol, available from Sasol North America, Inc.

### Microbes

Due to its low natural biodegradability, 1,4-dioxane has been detected in groundwater supplies in numerous areas. Several microbes, bacteria and fungi that have adapted to feed on and thus degrade 1,4-dioxane in aqueous solutions have been isolated. By way of example and not intended as a limitation, a representative list of microbes that therefore may be suitable for use in the degradation of dioxanes include the following: Agrocybe aegerita (see Kinney, et al., supra); Cordyceps sinesis (see Nakamiya, et al., supra); Burkholderia cepacia G4, Escherichia coli TG1 (T2MO), Escherichia coli TG1 (T4MO), Escherichia coli TG1 (TpMO), Methylosinus trichorsporium OB3b, Mycobacterium vaccae JOB5, Pseudomonas mendocina KR1, Pseudonocardia benzenivorans B5, Pseudonocardia K1, Ralstopnia picketti PK01, and Rhodococcus RR1 (see Mahendra, et al., supra); and Pseudonocardia dioxanivorans CB1190 (see Mahendra, et al., Parales, et al., and Alvarez, et al., supra).

The foregoing microbes have all been shown to degrade dioxane through mechanisms that are postulated to be enzymatic (see Mahendra, et al., and Nakamiya, et al., supra). The enzymes are assumed to be peroxygenases, monooxidases or etherases; in at least one case, the enzyme was extracellular. According to the present invention, enzymes that are responsible for degradation of 1,4-dioxane can be utilized through introduction of microbes via the various techniques as described above. In each method, it is anticipated that the microbe to be used is grown in such a manner as to preferably seek out and degrade the cyclic ether. It is further anticipated that some enzymes are capable of being isolated extracellularly, which isolated enzymes can be used either alone or in cooperation with a cofactor. Cofactors as used herein are understood to refer to non-protein chemical compounds that are bound to a protein and are required for biological activity of the protein. Cofactors may be either organic or inorganic. Examples of inorganic cofactors include metal ions and iron-sulfur clusters. Examples of organic cofactors include: vitamins and their derivatives; non-vitamins; as well as protein and non-protein molecules that either activate, inhibit or are required for the protein to function, such as hormones. Examples of cofactors that are appropriate for use with the inventive methods and compositions discussed herein may be found at
http://en.wikipedia.org/wiki/Cofactor_%28biochemistry%29 (3 Dec. 2012).

### Treatment Methods

One method for the removal of 1,4-dioxane from surfactants according to the instant invention therefore comprises treating liquid systems with enzymes or enzyme-producing microbes that are capable of degrading cyclic ethers. Alternately, such microbes may be pre-treated such that they express enzymes capable of degrading cyclic ethers. In another alternate approach, isolated enzymes may be used that are capable of degrading cyclic ethers, especially where the cyclic ether is preferably 1,4-dioxane. The liquid systems can include surfactant preparations for use as raw materials in cleaning products or actual cleaning product formulations.

The microbes or enzymes that are contemplated for use in degrading dioxanes or cyclic ethers according to the present invention are those that are capable of preferably degrading cyclic ethers over linear ethers. Examples of the latter include ethoxylated surfactants. The microbes or enzymes that are used for degrading cyclic ethers that are contemplated for use with the present invention should demonstrate a specificity for cyclic ethers over linear ethers in a ratio that is at least 2:1, preferably 10:1 and most preferably 100:1 cyclic ethers to linear ethers. The kinds of liquid systems that are anticipated to be suitable subjects for microbe treatment for the removal of dioxanes according to the present invention may also contain other ingredients such as non-ethoxylated surfactants, solvents, builders, dyes and colorants, optical brighteners, fragrances, and preservatives. If a liquid system also contains agents such as oxidants, reductants, and/or other enzymes, these substances should be able to retain at least 10%, preferably 50%, and most preferably 90% of their viability, relative to liquid systems without the addition of the cyclic ether-degrading enzymes or enzyme-producing microbes described herein.

In a method according to another embodiment of the invention, one or more microbes may be added to an aqueous solution of at least one ethoxylated surfactant to degrade 1,4-dioxane by at least 10% by weight, preferably at least 25% by weight, more preferably at least 50% by weight as compared to the starting amount of dioxane. Most preferably, the reduction of 1,4-dioxane would be lower than analyzable levels, that is below 1 part per million. Once a sufficient amount of 1,4-dioxane has been degraded, thus creating a purified ethoxylated surfactant solution, the purified ethoxylated surfactant solution may be added into a cleaning formulation.

In yet another embodiment of the invention, one or more microbes may be added to an aqueous solution cleaning composition containing 1,4-dioxane to degrade it by at least 10% by weight, preferably at least 25% by weight, more preferably at least 50% by weight of the original amount of dioxane. Most preferably, the reduction of dioxane would be lower than analyzable levels, that is, below 10 parts per billion. Once a sufficient amount of 1,4-dioxane has been degraded, creating a purified cleaning composition, the purified cleaning composition may be packaged and sold to consumers. Depending on the method of making the cleaning composition, one or more microbes may be added at one or more stages in the process of making the cleaning composition.

According to another embodiment of the invention, one or more microbes may be added to a finished cleaning composition at various stages in the supply chain. For example, dioxane-degrading microbes may be added to a finished cleaning composition by: a manufacturer, a co-packer, a consumer or industrial user, etc. Also contemplated as part of the present invention is that the microbes that are used for the degradation of dioxanes may be removed from raw materials, cleaning compositions or other suitable solutions following their use, which microbes may then be re-used in subsequent compositions. Alternatively, the methods contemplated herein for use in the degradation of dioxanes may permit the microbes to remain with the raw materials or finished composition.

### EXAMPLES

A sample of a microbe that was isolated from a sludge sample containing 1,4-dioxane and shown to grow in the presence of 1,4-dioxane as the sole carbon and energy source can be introduced to a sample of alkyl ether sulfate containing 50 ppm of 1,4-dioxane. After incubation at ambient temperature for three days, the level of 1,4-dioxane can be significantly reduced. The treated alkyl ether sulfate maintains its ability to generate foam as well as to clean soil samples.

A sample of an enzyme expressed from a microbe that was isolated from a sludge sample containing 1,4-dioxane and shown to grow in the presence of 1,4-dioxane as the sole carbon and energy source may be introduced into a sample of alkyl ether sulfate containing 50 ppm of 1,4-dioxane. After incubation at ambient temperature for three days, the level of 1,4-dioxane can be significantly reduced. The treated alkyl ether sulfate maintains its ability to generate foam as well as to clean soil samples.

The present invention has been described above in detail with reference to specific embodiments, methods and examples. However, these specific embodiments should not be construed as narrowing the scope of the invention, but rather construed as illustrative examples. It is to be further understood that obvious embodiments, modifications and equivalents thereof are anticipated and are considered to be within the scope of the claimed invention without departing from the broad spirit contemplated herein. The invention is further illustrated and described in the claims that follow.

1. A method for the degradation of cyclic ethers in a liquid stream, comprising adding to the liquid stream at least one of: a cyclic ether degrading enzyme, a cyclic ether degrading enzyme-producing microbe, and combinations thereof, wherein the liquid stream comprises at least one cleaning active, and wherein the cleaning active is selected from among the group comprising: anionic surfactants; nonionic surfactants; solvents; derivatives thereof; and mixtures thereof.

2. The method of claim 1 wherein the microbe is selected from among the group comprising: Agrocybe aegerita; Burkholderia cepacia G4; Cordyceps sinesis; Escherichia coli TG1 (T2MO); Escherichia coli TG1 (T4MO); Escherichia coli TG1 (TpMO); Methylosinus trichorsporium OB3b; Mycobacterium vaccae JOB5; Pseudomonas mendocina KR1; Pseudonocardia benzenivorans B5; Pseudonocardia dioxanivorans CB1190; Pseudonocardia K1; Ralstopnia picketti PK01; and Rhodococcus RR1.

3. The method of claim 1 wherein the enzyme is selected from among the group comprising: etherases, peroxygenases, monooxygenases, oxidases, and an etherase and an oxidase.

4. The method of claim 1 wherein:
a. the anionic surfactant is selected from among the group comprising alkyl ether sulfates, alkyl ether carboxylates, alkyl ether phosphates, derivatives of any of the foregoing, and combinations thereof; and wherein
b. the nonionic surfactant is selected from among the group comprising ethoxylates of linear and branched primary and secondary alcohols, amides, amines, derivatives of any of the foregoing, and mixtures thereof.

5. The method of claim 4 further wherein:
a. the anionic surfactant is selected from among the group comprising: a C₈₋₁₈ fatty alcohol, which has been ethoxylated with an average of about 1-20 moles of ethylene oxide per mole of alcohol, and subsequently sulfonated, carboxylated or phosphated; and
b. the nonionic surfactant is selected from among the group comprising: C₆₋₁₈ alcohols with 1-15 moles of ethylene oxide per mole of alcohol; C₆₋₁₈ alcohols with 1-15 moles of ethylene oxide and 1-10 moles of propylene oxide per mole of alcohol; C₆₋₁₈ alkylphenols with 1-15 moles of ethylene oxide, propylene oxide, or ethylene oxide and propylene oxide per mole of alcohol; derivatives of any of the foregoing; and mixtures thereof.

6. The method of claim 1 wherein the enzyme further comprises at least one organic or at least one inorganic cofactor, wherein
a. the organic cofactor is selected from among the group comprising: vitamins; non-vitamins; protein and non-protein molecules; derivatives of any of the foregoing; and combinations thereof; and
b. the inorganic cofactor is selected from among the group comprising: metal ions; iron-sulfur clusters; derivatives of any of the foregoing; and combinations thereof.

7. The method of claim 1 wherein the enzyme or enzyme-producing microbe demonstrates a specificity for cyclic ethers over linear ethers in a ratio that is at least 2:1, preferably 10:1 and most preferably 100:1 cyclic ether to linear ether.

8. The method of claim 1 wherein the liquid system retains at least 10%, preferably 50%, and most preferably 90% of its viability, relative to liquid systems without the addition of the cyclic ether degrading enzymes or enzyme-producing microbes described herein.
